(19) United States
(12) Patent Application Publication
(10) Pub. No.: US 2004/0072199 A1
(43) Pub. Date: Apr. 15, 2004

(54) Method for marking samples containing dna by means of oligonucleotides

(76) Inventor: Gottfried Brem, Hilgertshausen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/416,122

(22) PCT Filed: Nov. 7, 2001
(86) PCT No.: PCT/EP01/12880

(30) Foreign Application Priority Data
Nov. 8, 2000 (DE) 10055368.0

(51) Int. Cl. C12Q 1/68; C12P 19/34

(52) U.S. Cl. 435/6; 435/91.2

(57) ABSTRACT

The invention relates to a method for marking samples containing DNA. At least one oligonucleotide marker is associated with the sample, and the sample is then analysed together with the oligonucleotide marker. Said oligonucleotide marker is selected from the group consisting of artificial microsatellite oligonucleotides or artificial oligonucleotides of single nucleotide polymorphisms.

The present invention relates to a method for the identification of DNA containing samples, wherein at least one oligonucleotide as an internal identification means is brought into contact with the sample and subjected to a subsequent examination together with the sample. The oligonucleotide is selected from the group consisting of artificial microsatellite oligonucleotides or artificial oligonucleotides of single nucleotide polymorphisms.

The increasing importance of molecular genetics and the accompanying increasing extent of laboratory diagnostic examinations has led to a more and more increasing number of samples being collected, transported, stored and analyzed. Thereby, the problem of a mixing-up of the samples or of a loss of the identity arises due to a loss or an illegibility of the identification. In particular, mixing-up may occur during the collection and storage of samples in the framework of mass screening or in the preparation of genetic resource collections, rendering vain the measures which have been taken. As a result, enormous costs may arise and values get lost, respectively.

Currently, in many fields of the daily live samples are collected, gathered and stored for a later examination, such as for example, for the identification/typing of animals, monitoring of foodstuffs and in human and veterinary medicine, etc. In all these cases it is imperative that a reliable individual identification is carried out for each obtained sample.

At present, this is generally achieved by identifying the containers, in which the samples will be introduced, e.g. by a barcode or simply by hand, and the container is assigned to an individual. This kind of identification has however drawbacks since the identification on the container may get lost or become illegible and is only as long associated with the samples as these are present in the containers.

Several methods have been proposed in the state of the art in order to overcome these drawbacks. For example, WO 96/17954 discloses a method for chemical identification of an object, wherein according to the invention at least two chemical markers are used. One marker shows that the container itself has been marked, while the other marker is in principle the real identification.

Furthermore, in the U.S. Pat. No. 5,776,737 a method for the identification of samples is disclosed, wherein oligonucleotides are added to the sample obtained, which will be sequenced together with the sample after a subsequent amplification step. The oligonucleotides consist of a primer binding site and an identification region consisting of an alternating sequence of nucleotides (MN)ₓ and (MNN)ₓ, respectively, wherein N is the nucleotide of the primer binding site. The sample can be identified by sequencing the identification region. A drawback of this method is however that the chosen oligonucleotides, in particular the primer binding site, may not contain any sequences occurring in the individual itself, as otherwise endogenous sequences would interfere during the sequencing of these identification oligonucleotides.

Therefore, the object of the present invention is to provide an alternative and simplified method for the identification of samples, which overcomes the disadvantages present in the state of the art.

This object is solved by a method, wherein a sample is collected from an individual, said sample is brought together with an identification oligonucleotide and said sample is then subjected to an examination together with the identification oligonucleotide, wherein said identification oligonucleotide is selected from the group consisting of artificial microsatellite oligonucleotides (AMS oligonucleotides) or artificial oligonucleotides of single nucleotide polymorphisms (ASNP oligonucleotides).

An advantage of the present invention is that the decodification of the identification oligonucleotides occurs during the same step and with the same detection method as the examination of the sample DNA and can be performed without time and cost intensive sequencing methods. Hereby, not only the working processes are simplified, but also sources of error are excluded, which may result in spite of usual precautionary methods during two work steps. Moreover, it is also possible according to the present invention, to use endogenous sequences within the oligonucleotides, rendering their selection more easy and reducing the error rate.

In the Figures, wherein:

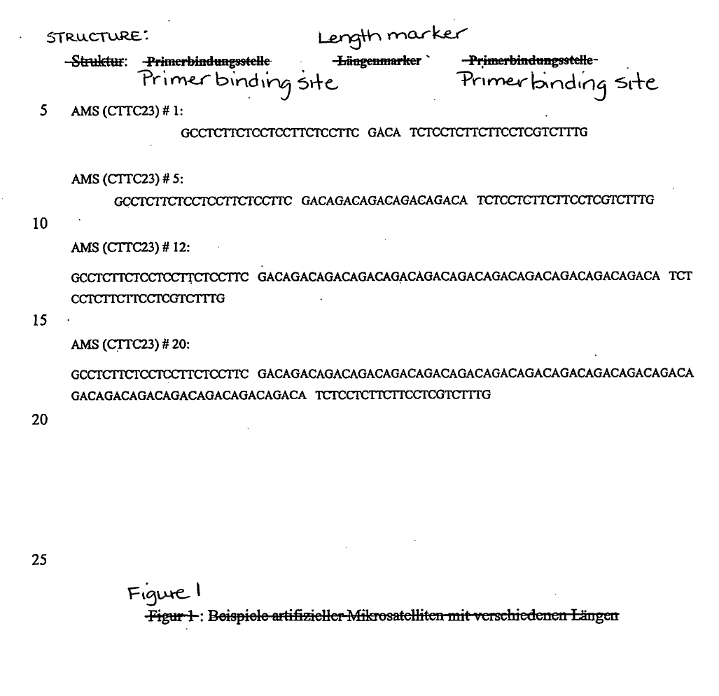

FIG. 1 shows artificial microsatellites for the production of AMS types for the purpose of sample individualization. Four examples of a set of exemplarily chosen 20 lengths are shown here: AMS (CTTC23)#1, AMS (CTTC23) #5, AMS (CTTC23) #12, AMS (CTTC23)#20.

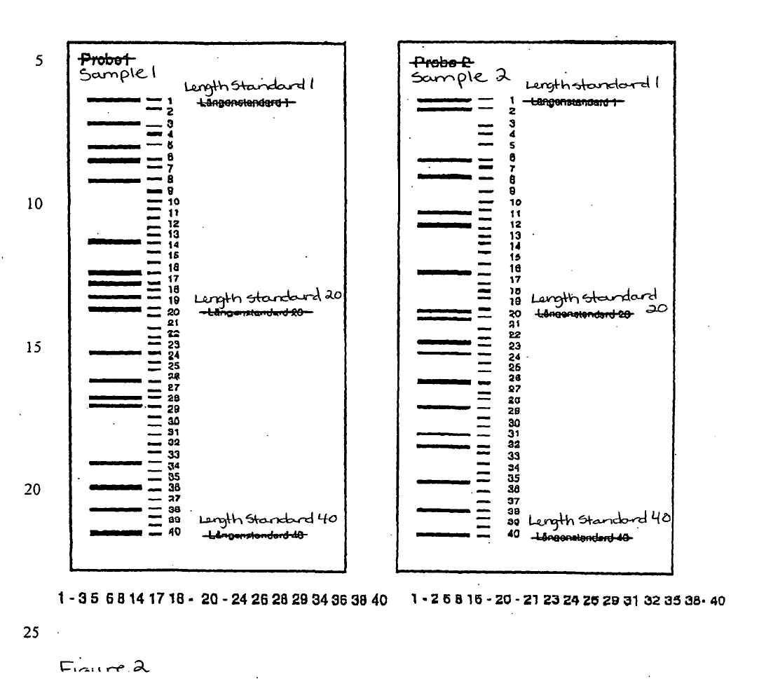

FIG. 2 schematically shows on the basis of two samples the use of an oligocode with three length standards and 37 variables, which is sufficient for the typing of 137 million individual samples. As may be seen from FIG. 2, the three length standards #1, #20 and #40 are contained in all the samples.

The sample collecting means used according to the present invention, are selected from the group consisting of artificial microsatellite oligonucleotides (AMS oligonucleotides) or artificial oligonucleotides of single nucleotide polymorphisms (ASNP oligonucleotides).

According to the present inventions, artificial microsatellite oligonucleotides (AMS) are oligonucleotides containing two specific flanking sequences (identical or different on the 3′ and 5′ end) that contain in between them a uniform DNA sequence of fixed length, having at least one base (the use of tetramers results in easily interpretable results). The flanking sequences serve during the decodification as primer binding sites (PBS) for the PCR amplifications and have a length, which permits a hybridization with complementary oligonucleotides under the respective chosen conditions, for example between 10 and 15 bp. FIG. 1 shows several artificial microsatellites with internal lengths.

An individuality identification will now be obtained by different combinations of AMS oligonucleotides. In a first step, e.g. 40 different AMS oligonucleotides are synthesized. A computer-controlled device is then loaded with these 40 AMS oligonucleotides and according to an EDP program pipettes together individual identifications from these AMS by bringing together different combinations of these 40 AMS oligonucleotides, i.e. specific AMS are pipetted and others are omitted. These AMS oligonucleotide mixtures are introduced either directly in a receptacle subsequently used as a sample container that is either pre labeled or will be marked during filling, or stored temporarily in a storage container with identification. The connection between AMS-type and directly readable identification is performed by an EDP program.

By the different combinations of these artificial microsatellites one can attain an extremely high variability. By combining, e.g., 64 different AMS more than 2⁶⁴ (>18 trillion) individual combinations may be generated. For providing individual AMS for all economically useful animals currently living on earth only a combination of 32 different AMS oligonucleotides is required. The oligonucleotides required for this purpose may be easily and inexpensively synthesized.

When filling up the sample containers, the AMS type, i.e. the specific mixture of the AMS oligonucleotides, comes into direct contact with the sample and gets mixed with it. In biological samples, the longevity of the oligonucleotides is at least equivalent to that of the sample (DNA) itself. Additionally, the oligonucleotides can be placed on objects, in which case the stability of the identification oligonucleotides will depend on the material and the treatment.

For screening purposes, during the analysis, the presence of AMS oligonucleotides can be determined relatively easily and economically, for example by performing a PCR with primers complementary to the PBS and separating the so obtained fragments and demonstrating them in an appropriate manner. The resulting pattern (see FIG. 2) is unique and permits the assignment of the sample identity to an individual.

In order to increase the certainty of the evaluation, length standards can be used, which are alleles occurring in each AMS type and thus indicating by their presence during the detection that the PCR functioned properly and forming at the same time a length standard, wherein one AMS is the shortest, one is the longest possible and one lies exactly in the middle. Even further certainty can be provided by including into each AMS mixture a mixture of two different PBS, which therefore will be amplified with different primers. A comparison of the two patterns which have to be identical, confirms the correctness and provides an additional security. Should this statement of security still not be sufficient, a third AMS locus can be used, which comprises two alleles which stand for the number of present and for the number of missing alleles in the AMS types (for example, in sample 1 in FIG. 2, 18 alleles are present and 22 are missing).

As outlined above, the detection may be performed by means of PCR amplification. A sequencing is not necessary. Depending on the application, it is additionally possible to directly detect previously introduced AMS oligonucleotides, i.e. to detect without amplification the length polymorphism of DNA fragments by gel electrophoresis, capillary electrophoresis, mass spectroscopy or a comparable procedure. The detection may be performed very quickly (i.e. in less than 1 hour inclusive isolation) and economically and may be performed together with the detection of the sample itself.

According to another embodiment, it is possible to directly encode a code in DNA fragments, for example a barcode or a combination of characters. According to the present invention, this is accomplished with artificial oligonucleotides of single nucleotide polymorphisms (ASNP oligonucleotides).

According to the present invention, ASNP oligonucleotides are oligonucleotides which differ at a specific position of the oligonucleotide. These ASNP oligonucleotides are designed in such a way that a specific nucleotide which is present either at, an end of or within the oligonucleotide, alternatively is either a C or T and a A or G, respectively. In this way with one oligo three distinguishable types may be given (as an example of a polymorphism at an end position).

| | | |
|---|---|---|
| Homozygous type CC | | |
| GCC TCT TCT CCT CCT TCT CCT TCC | or | Oligo |
| | abbreviated | 1-C |
| GCC TCT TCT CCT CCT TCT CCT TCC | | Oligo |
| | | 1-C |
| Heterozygous type CT | | |
| GCC TCT TCT CCT CCT TCT CCT TCC | | Oligo |
| | | 1-C |
| GCC TCT TCT CCT CCT TCT CCT TCT | | Oligo |
| | | 1-T |
| Homozygous type TT | | |
| GCC TCT TCT CCT CCT TCT CCT TCT | | Oligo |
| | | 1-T |
| GCC TCT TCT CCT CCT TCT CCT TCT | | Oligo |
| | | 1-T |

If several different oligonucleotides are used, according to the formula 3′ e.g. 10 different oligonucleotides codify 59049 types. As a result, artificial identifications may be performed when using oligonucleotide sequences occurring in the DNA of the species from which the sequence has been derived and which do not have any variability at this position or which have an endogenous A or G variability.

When using oligonucleotides, the sequence of which does not occur in the species, also the other two nucleotides may be used. As a result, three additional types result with the same oligonucleotide but the other nucleotide pair:

| | | |
|---|---|---|
| Homozygous type AA | | |
| TCT CCT CTT CTT CCT CGT CTT TG A | or | Oligo |
| | abbreviated | 1-A |
| TCT CCT CTT CTT CCT CGT CTT TG A | or | Oligo |
| | abbreviated | 1-A |
| Heterozygous type AG | | |
| TCT CCT CTT CTT CCT CGT CTT TG A | | Oligo |
| | | 1-A |
| TCT CCT CTT CTT CCT CGT CTT TG G | | Oligo |
| | | 1-G |
| Homozygous type GG | | |
| TCT CCT CTT CTT CCT CGT CTT TG G | | Oligo |
| | | 1-G |
| TCT CCT CTT CTT CCT CGT CTT TG G | | Oligo |
| | | 1-C |

From a combination of these oligonucleotides with 4 different nucleotides when diallelically used (i.e. two oligonucleotides per sample) a total of 10 different combinations may be obtained:

AA, AC, AG, AT, CC, CG, CT, GG, GT, TT.

Therefore it is possible to transform each number into a DNA code by defining oligonucleotides which stand for the units digit, tens digit, hundreds digit, thousands digit, etc., for example:

Oligonucleotide for units digit:
TCT CCT CTT CTT CCT CGT CTT TG A-variable C, G
or T
Oligonucleotide for tens digit:
CCT GCT CTT CTT GTC TCT TCT CTG A-variable C, G
or T
Oligonucleotide for hundreds digit:
GCT TGT CCT CTG TTC TTT GTT TCG C A-variable C, G
or T
Oligonucleotide for thousands digit:
CCT CTT CGC TCT CTT GCT CTG CTC CT A variable C, G
or T

In addition to the different sequence, the oligos may also be designed having a variable length.

For a codification of digits always the same combination of bases is used (the variable position with which position the digits are codified may be provided at any position of the oligonucleotides, i.e. also in the center).

For example the following codification for the digits may be used:

1 2 3 4 5 6 7 8 9 10
AA AC AG AT CC CG CT GG GT TT

According to this system, e.g. the number 2103 is coded by the following oligonucleotide combinations:

| | |
|---|---|
| 2000: | CCT CTT CGC TCT CTT GCT CTG CTC CT-A |
| | CCT CTT CGC TCT CTT GCT CTG CTC CT-C |
| 100: | GCT TGT CCT CTG TTC TTT GTT TCG C-A |
| | GCT TGT CCT CTG TTC TTT GTT TCG C-A |
| 00: | CCT GCT CTT CTT GTC TCT TCT CTG-T |
| | CCT GCT CTT CTT GTC TCT TCT CTG-T |
| 3: | TCT CCT CTT CTT CCT CGT CTT TG-A |
| | TCT CCT CTT CTT CCT CGT CTT TG-G |

Any number of 4 digits can thus be represented with 8 different oligonucleotides. For a number of 7 digits, 14 oligonucleotides would be needed accordingly. By this coupling of a number with an oligonucleotide, a number associated with the sample, e.g. printed on the ear tag may be directly converted into an oligo-code. As a result, the ear tag number and the sample present in the corresponding container are inseparably associated one with another.

From genetics, a number of DNA polymorphisms are known, such as satellite DNA or SNPs which are also exploited for typing of individuals. At each gene locus, with the exception of the gonosomes or in case of chromosomal aberrations, each individual has two alleles. These alleles may be identical or different. By conducting an analysis of the alleles at several up to many genetic loci, a characteristic pattern for each individual, a genotype, will be obtained, which characterizes this animal unmistakeably. Each animal may have at one locus always maximally only two different alleles, but in the population many different alleles may occur at one gene locus (multiple alleles). This polymorphism forms the basis for the DNA individuality of organisms and can be used for the identification of individuals.

In the analysis of a tissue/DNA sample of an individual for ASNP genotypes, the ASNP oligonucleotides which codify the number are automatically and concomitantly analyzed using the same method as for identifying the endogenous SNPs. The costs for the analysis of identity numbers are negligibly low due to the identical detection method. In a currently used SNP analysis of a cow, about 1-200 SNPs are analyzed. With only ten percent of this number, a ear tag number may be concomitantly identified and thus from the result of the SNP analysis not only the genotype of the animal at the SNP loci may be determined, but quasi simultaneously its ear tag number may be read. By comparing this DNA internal number with the given number of the animal from which the sample has been taken, it may be immediately determined whether this indication is correct or if the sample has been obtained from another animal. As the assignment of the oligonucleotides to the digits may be freely chosen, a forging may be prevented when keeping this assignment secret.

Loading the sample containers is carried out in such a way that a computer-aided device pipettes the combination for the tens digits, hundreds digits and thousands digits accordingly and adds then for each consecutive number the two oligonucleotides for the units digits. For the next step of tens, hundreds, etc. the stock mixture is prepared accordingly and used. As a result, the pipetting expenses per collection container are kept low and the operation may be performed quickly. Additionally, no extra record keeping and e.g., electronic data combination has to be performed, respectively, as the DNA code according to the assignment may be later directly read from the ASNPs.

A system which is particularly appropriate for the present invention is described in the WO 99/61822, which is herewith incorporated by reference. In case of the ear tag disclosed in this pamphlet, the oligonucleotides may be introduced in the hollow tip of the ear tag spike, and if necessary, this one may be provided with a protective layer, in order to avoid contamination. During the sample collection, which comprises for example puncturing of an ear of an economically useful animal, the oligonucleotides present in the ear tag spike come into contact with the sample, so that the sample may always be identified on basis of the oligonucleotides. Equally, the oligonucleotides may be previously given in the sample container.

According to an embodiment, the sample container may also contain a strongly hygroscopic compound, as described in DE 199 57 861.3, in order to increase the stability during storage of the sample.

Furthermore, the present invention may also be used in a process for examining the individuals of a population, wherein the genomic DNA of the individuals is fixed on a matrix, so that to each individual a specific identifiable segment on the matrix may be assigned (see DE 100 00 001). During the sample collection, an identification oligonucleotide is added to the DNA to be fixed on the matrix, which is fixed simultaneously on the matrix. Subsequently, it may always be determined via the identification oligonucleotides which segment is assigned to a specific individual.

The following example illustrates the advantages of the present invention and should not be construed to limit the scope of the present invention.

### EXAMPLE

A system developed for the sample collection from economically useful animals which allows during the taking of the sample tissue simultaneously obtaining DNA containing samples and which is disclosed in the WO 99/61822, has been used to take samples from 10 cows. On basis of the system described in the above indicated WO publication, an identification of the cows with a simultaneously occurring corresponding identification of the sample containers could be performed, wherein pre-lettered parts for the ear tag and the container have been used, respectively.

Subsequently, different mixtures (100 pg) of previously prepared identification oligonucleotides have been introduced into the containers, which were associated with the markings associated with the ear tags and the containers, and the containers were stored for 1 week at −80° C.

Next, samples are taken from two containers and subjected to an amplification by means of PCR (reaction volume 15 μL, 0.5 μmoles primer, 0.2 μmoles dNTPs, 2.5 U Taq (hotstart polymerase from Applied Biosystems); 30 cycles; annealing at 60° C., 30 sec; reaction at 72° C. for 120 sec; denaturation at 95° C. for 30 sec) and the so obtained fragments were separated on a polyacrylamide gel (6%). FIG. 2 shows schematically the results of the separation. On basis of the previously stored assignment in the computer to a container/to an ear tag/to a cow, the coding could be performed without giving rise to any problems.

1. A method for identification of samples containing DNA, wherein at least one identification oligonucleotide and a sample to be identified are brought into contact, and said sample is subjected to a PCR examination together with the identification oligonucleotide, wherein the identification oligonucleotide is an artificial microsatellite oligonucleotide.

2. The method of claim 1, wherein the artificial microsatellites have a nucleotide sequence of a fixed length of repetitive nucleotide sequences.

3. The method according to any of the preceding claims, wherein the sample is introduced into a container containing an identification oligonucleotide.

4. The method according to any of the preceding claims, wherein the oligonucleotides are introduced in the hollow tip of an ear tag spike, are provided with a protective layer and come into contact with it when obtaining the sample.

5. The method according to any of the preceding claims, wherein the identification oligonucleotides are assigned to a numerical or alphanumerical system.


SEQUENCE LISTING (14 sequences)

SEQ ID NO: 1 - DNA, 24 bp, Artificial Sequence
  synthetic oligonucleotide
gcctcttctcctccttctccttcc

SEQ ID NO: 2 - DNA, 24 bp, Artificial Sequence
  synthetic oligonucleotide
gcctcttctcctccttctccttct

SEQ ID NO: 3 - DNA, 24 bp, Artificial Sequence
  synthetic oligonucleotide
tctcctcttcttcctcgtctttga

SEQ ID NO: 4 - DNA, 24 bp, Artificial Sequence
  synthetic oligonucleotide
tctcctcttcttcctcgtctttgg

SEQ ID NO: 5 - DNA, 25 bp, Artificial Sequence
  synthetic oligonucleotide
cctgctcttcttgtctcttctctga

SEQ ID NO: 6 - DNA, 26 bp, Artificial Sequence
  synthetic oligonucleotide
gcttgtcctctgttctttgtttcgca

SEQ ID NO: 7 - DNA, 27 bp, Artificial Sequence
  synthetic oligonucleotide
cctcttcgctctcttgctctgctccta

SEQ ID NO: 8 - DNA, 27 bp, Artificial Sequence
  synthetic oligonucleotide
cctcttcgctctcttgctctgctcctc

SEQ ID NO: 9 - DNA, 25 bp, Artificial Sequence
  synthetic oligonucleotide
cctgctcttcttgtctcttctctgt

SEQ ID NO: 10 - DNA, 24 bp, Artificial Sequence
  synthetic oligonucleotide
tctcctcttcttcctcgtctttgg

SEQ ID NO: 11 - DNA, 50 bp, Artificial Sequence
  artificial microsatellite
gcctcttctcctccttctccttcgacatctcctcttcttcctcgtctttg

SEQ ID NO: 12 - DNA, 66 bp, Artificial Sequence
  artificial microsatellite
gcctcttctcctccttctccttcgacagacagacagacagacatctcctcttcttcctcgtctttg

SEQ ID NO: 13 - DNA, 94 bp, Artificial Sequence
  artificial microsatellite
gcctcttctcctccttctccttcgacagacagacagacagacagacagacagacagacagacagacagacatctcctcttcttcctcgtctttg

SEQ ID NO: 14 - DNA, 126 bp, Artificial Sequence
  artificial microsatellite
gcctcttctcctccttctccttcgacagacagacagacagacagacagacagacagacagacagacagacagacagacagacagacagacagacagacagacatctcctcttcttcctcgtctttg